(19) United States
(12) Patent Application Publication
(10) Pub. No.: US 2006/0155281 A1
(43) Pub. Date: Jul. 13, 2006

(54) Device for fixing bones

(76) Inventors: Thomas Kaup, Davos Platz (CH); Chad Munro, Mabou (CA)

Correspondence Address: JONES DAY, 222 EAST 41ST STREET, NEW YORK, NY, 10017-6702, US

(21) Appl. No.: 10/529,981

(22) PCT Filed: Oct. 1, 2002
(86) PCT No.: PCT/CH02/00542

§ 371 Date: Dec. 16, 2005

(51) Int. Cl. A61F 2/30

(52) U.S. Cl. 606/65

(57) ABSTRACT

The invention relates to a device for fixing bones, comprising: A) an intramedullary nail (1) having at least one cross hole (2) with a central axis (3), and; B) a bone fixation means (4) with a front end (6) and with a rear end (5) that is intended for inserting into the cross hole (2) of the intramedullary nail (1), whereby; C) the bone fixation means (4) can be elastically expanded.

The invention relates to a device for fixing bones of the introductory portion of claim 1 and to an elastically expandable sheath of claim 23.

Medullary pins, intended for the femur, frequently are configured with proximal locking, that is, with at least one screw or one bone blade, introduced transversely to the longitudinal axis of the medullary pin. Such a medullary pin, with an elongated hole at the proximal end, extending transversely to the longitudinal axis of the medullary pin for accommodating bone screws or bone blades, is known from the EP 0 613 663 of FRIGG. The use of a bone screw or bone blade, disposed transversely to the longitudinal axis, in the event of a fracture of the neck of the femur, is disadvantageous because the head of the hip joint can rotate relative to the bone screw or the bone blade.

The invention is to provide a remedy here. It is an object of the invention to create a bone screw with a sheath, the configuration of which can be varied during the fixing in the head of the hip joint, so that the sheath no longer is rotationally symmetrical and therefore a rotation of the head of the hip joint relative to the sheath is precluded.

Pursuant to the invention, this objective is accomplished with a device for fixing bones, which has the distinguishing features of claim 1, as well as with an elastically expandable sheath, which has the distinguishing features of claim 23.

The inventive device comprises essentially an intramedullary pin with a transverse borehole and a bone fixation agent, which can be introduced with its rear end into the transverse borehole in the medullary pin. The bone fixation agent is flexible at least at its front end and can be expanded elastically transversely to the central axis of the transverse borehole.

The inventive device can be used in porous tubular bones and has the following advantages:
The flexibility of the bone-fixation agent promotes the formation of bony tissue (callus);
In the event that the implant is overloaded, the flexibility of the bone fixation agent enables the implant to move together with the porous bone without severing it. After the stress is relieved, the flexible element of the bone-fixation agent, together with the bone, assumes the initial position once again;
The flexible part of the bone fixation agent is configured so that, as is also the case in the state of the art, it absorbs the load partly to fully for most types of fractures; however, it has the above-mentioned flexibility.
The expansion of the interface between bone and implant increases the resistance to the implant being pulled out (cut-out); and
The expansion of the flexible part of the bone fixation agent makes a better hold possible because of the compression of the bone around the bone fixation agent.

In the preferred embodiment of the inventive device, the bone fixation agent comprises at its rear end a cylindrical or prismatic shaft, which is coaxial with the central axis of the transverse borehole and, at the front end, an elastically expandable sheath, which is also coaxial with the central axis of the transverse borehole. The sheath and the shaft may be constructed in one piece or, in a different embodiment of the inventive device, may be separate parts, which can be assembled coaxially, whereby the advantage can be achieved that the shaft and the sheath can be produced from different materials. The connection between the shaft and the sheath can be established as a screw connection, a conical connection or also as a compression joint. The following advantages can be attained by this configuration of the bone fixation agent:
The expansion of the sheath increases the contact area between the bone and the implant at the end and side surfaces of the sheath, so that smaller contact stresses can be achieved;
The expansion of the sheath changes its shape, so that it is no longer symmetrical and therefore a notation of the head of the hip joint relative to the sheath is precluded.

In a further embodiment of the inventive device, a second expandable sheath is disposed also coaxially with the central axis of the transverse borehole over the first inner sheath. As a result, the advantage can be attained that two functions, which usually must be borne by a single part, can be distributed in this way between two separate parts in the following manner:

1) the inner sheath primarily must absorb the load, which acts on the head of the femur and divert it to the femur and should therefore consist of a rigid material (such as titanium), whereas

2) the outer sheath functions as a connecting element between the bone, especially the spongiosa, and the inner sheath. The outer sheath has the task of ensuring an optimum introduction of force from the relatively soft bone to the hard, rigid implant and is preferably produced from a plastic (such as an elastomer), which has a modulus of elasticity, which approximates or is even clearly lower than the modulus of elasticity of the spongiosa. With that, on the one hand, an improved surface contact at the transition between implant and bone can be brought about and, on the other, a reduction in the maximum stresses occurring during a load, can be attained.

Depending on the material selected for the two sheaths, the second sheaths can be fastened to the first sheath by being sprayed, pressed, screwed or glued onto it.

In a different embodiment of the inventive device, the second sheath is configured with an external thread.

In yet another embodiment of the inventive device, the second sheath is configured as a blade, preferably as a spiral blade.

In another embodiment of the inventive device, the sheath has an external thread with a thread pitch of between 0.1 mm and 1000 mm.

In yet a different embodiment of the inventive device, the sheath is constructed as a blade.

In a further embodiment of the inventive device, the latter includes a safeguard against rotation, by means of which the bone fixation agent can be fixed in the transverse borehole of the medullary pin, so that it cannot rotate about the central axis of the transverse borehole.

In yet a further embodiment of the inventive device, the expansion agents penetrate the bone fixation agent coaxially and include, at the front end of the bone fixation agent, a cone, which tapers towards the rear end of the bone fixation agent. Instead of a cone, which tapers towards the rear end, it is also possible to have a cone, which tapers towards the front end. In other embodiments, the cone can also be mounted at the inner wall of the sheath.

The sheath is divided by at least once slot into two or more tongue-shaped, axially extending elements. The slot, which separates these tongue-shaped elements from one another, also enables the elements to move radially relative to one another, as a result of which the rotational flexibility of the sheath is produced. The distance between the two tongue-shaped elements can be increased by means of an expansion agent, which expands the tongue-shaped elements. The expansion of the two elements leads to compressible forces on the boundary between sheath and bone and changes the shape of the external thread, so that the latter no longer is rotationally symmetrical, as a result of which the bone material is compressed.

Further advantageous developments of the invention are characterized in the dependent claims.

The invention and further developments of the invention are explained in even greater detail in the following by means of partly diagrammatically representations of several examples, of which

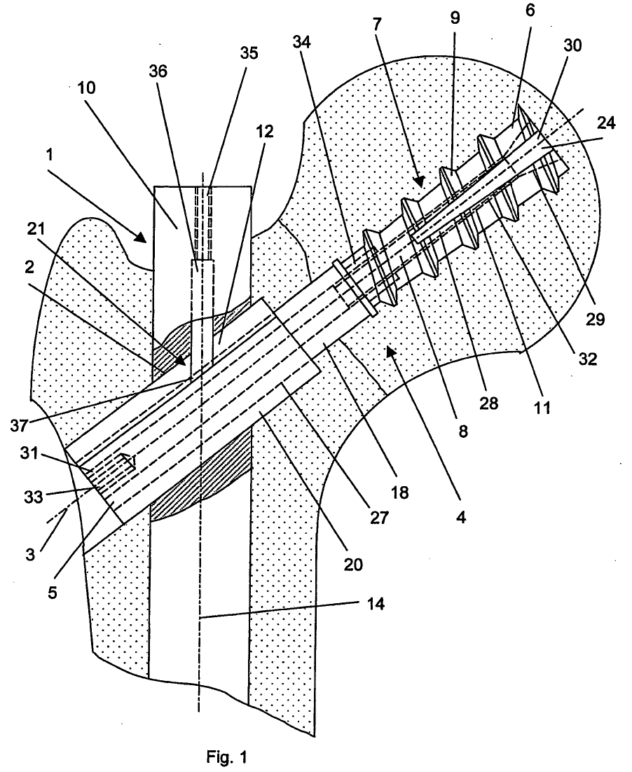

FIG. 1 is a view of an embodiment of the inventive device used at the proximal femur,

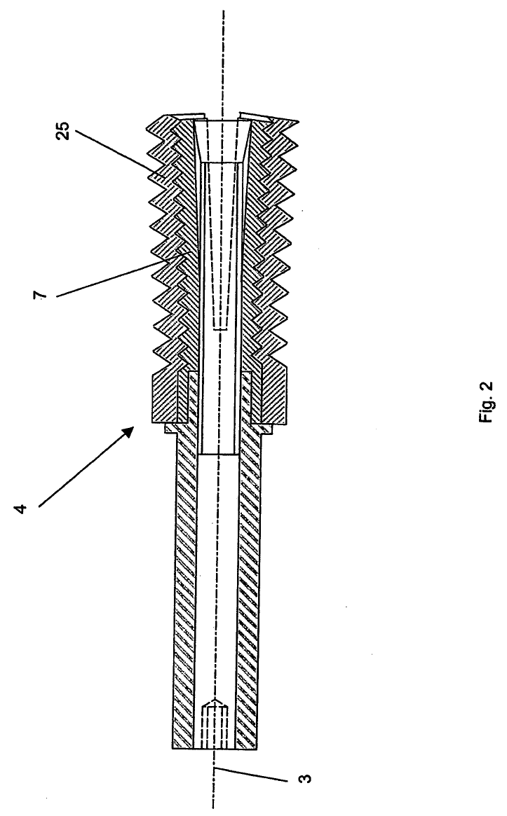

FIG. 2 represents a longitudinal section through an embodiment of a bone fixation agent with two concentrically disposed, expandable sleeves of an embodiment of the inventive device and

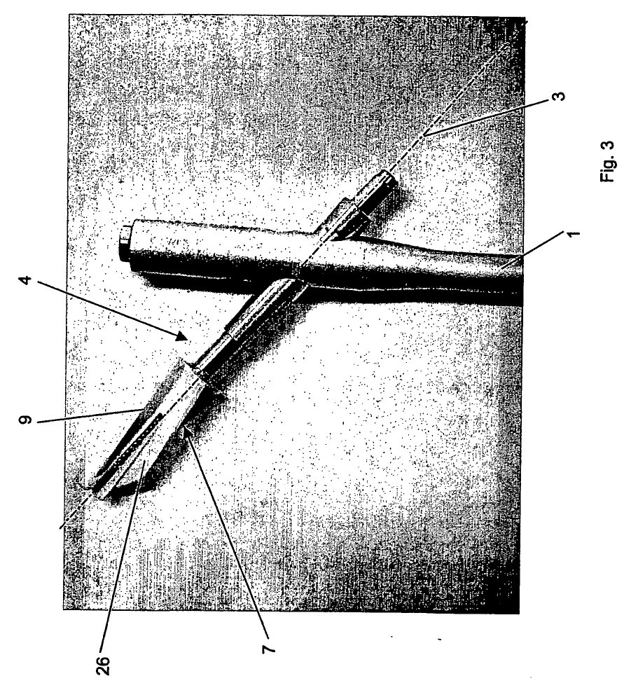

FIG. 3 shows a view of a further embodiment of the inventive device.

In FIG. 1, an embodiment of the inventive device is shown, which includes an intramedullary pin 1 with a longitudinal axis 14, as well as a bone fixation agent 4 configured as a bone screw. The medullary pin 1 is provided at its proximal end 10 with a transverse borehole 2, the central axis 3 of which encloses an angle α of between 125° and 135° with the longitudinal axis 14 of the medullary pin 1. A sliding sheath 20, in the central borehole 27 of which the rear end 5 of the bone fixation agent 4 is introduced and which is parallel to the central axis 3 of the transverse borehole 2, is introduced into this transverse borehole 2 coaxially with the central axis 3. At its front end 6, the bone fixation agent 4 includes a sheath 7, which is disposed coaxially with the central axis 3, can be expanded elastically transversely to the central axis 3 and has an external thread 9, which can be screwed into the bone. At its rear end 5, the bone fixation agent 4 includes a shaft 18, which is mounted in the central borehole 27 of the sliding sheath 20 parallel to the central axis 3 of the transverse borehole 2. Furthermore, the device includes an expansion agent 8, which can be moved parallel to the central axis 2, for expanding the sleeve 7. The expansion agent 8 consists of a screw 28, which can be screwed in to the internal thread 32 in the central borehole 11 of the sheath 7 and includes a cone 29 at the tip 30 of the screw. The cone 29 tapers towards the rear end 31 of the screw 28 and, when the screw 28 is screwed back, is retracted into the central borehole 11 of the sheath 7, so that the sheath 7 is expanded transversely to the central axis 3 in this way. The screw 28 penetrates through the shaft 18 of the bone fixation agent 4 up to the rear end 5 of the latter and can be rotated by means 33 for accommodating a screwdriver, which are provided at the rear end 31 of the screw 28. The means 33 for accommodating a screwdriver may be configured, for example, as a hexagon socket, a torx or a slot. The sheath 7 and the shaft 18 of the bone fixation agent 4 are disposed coaxially with the central axis 3 and connected with one another by means of a press connection 34. The sheath 7 has a slot 24, which is disposed so as to penetrate from the front end 6 of the bone fixation agent 4 transversely to the central axis 3 of the transverse borehole 2, by means of which the expandability of the sheath 7 can be attained. The shaft 18 of the bone fixation agent 4, as well as the central borehole 27 of the sliding sheath 20 have a noncircular cross-sectional area orthogonal to the central axis 3 of the transverse borehole, for example, a circular area, which is flattened at two sides. By these means, the bone fixation agent 4 is secured against rotating relative to the sliding sheath 20 about the central axis 3 of the transverse borehole 2. By means of a rotation safeguard 21, which can be tightened from the proximal end 10 of the medullary pin 1, the sliding sheath 20 can also be secured against rotating about the central axis 3 of the transverse borehole 2, while the bone fixation agent 4 remains movable parallel to the central axis 3 of the transverse borehole 2. The rotation safeguard 21 consists essentially of a screw 35 with a fork 36, which is parallel to the longitudinal axis 14 of the medullary pin 1 and the front ends 37 of which engage grooves 12 on the sliding sheath 20, which are parallel to the central axis 3 of the transverse borehole 2.

The embodiment of the bone fixation agent 4, shown in FIG. 2, differs from that shown in FIG. 1 in that a second sheath 25, which can also be expanded elastically transversely to the central axis 3 of the transverse borehole 2, is screwed with an external thread over the sheath 7.

The embodiment of the inventive device, shown in FIG. 3, differs from that shown in FIG. 1 only in that the sheath 7 is constructed as a spiral blade 26.

1. Device for fixing bones with
A) an intramedullary pin (1) with at least one transverse borehole (2) with a central axis (3) and
B) a bone fixation agent (4) with a front end (6) and a rear end (5) intended for introducing the medullary pin (1) into the transverse borehole (2), characterized in that
c) the bone fixation agent (4) can be expanded elastically.

2. The device of claim 1, characterized in that the bone fixation agents (4) include a central axis (3), a cylindrical all prismatic shaft (18) coaxial at the rear end (5) and a sheath (7), which can be expanded elastically, coaxially at the front end (6).

3. The device of claim 2, characterized in that a second, expandable sheath (25) is disposed on the first sheath (7) coaxially with the central axis (3).

4. The device of claims 2 or 3, characterized in that the sheath (7) is produced from a metal, preferably from titanium.

5. The device of claims 3 or 4, characterized in that the second sheath (25) is produced from a plastic, preferably from an elastomer.

6. The device of claim 5, characterized in that the second sheath (25) is sprayed onto the first sheath (7).

7. The device of one of the claims 3 to 5, characterized in that the second sheath (25) can be pressed onto the first sheath (7).

8. The device of one of the claims 3 to 5, characterized in that the second sheath (25) can be screwed onto the first sheath (7).

9. The device of one of the claims 3 to 5, characterized in that the second sheath (25) can be glued onto the first sheath (7).

10. The device of one of the claims 2 to 9, characterized in that the shaft (18) and the sheath (7) are separate parts, which can be connected coaxially with one another.

11. The device of one of the claims 1 to 10, characterized in that it includes a rotation safeguard (21), by means of which the bone fixation agents (4) can be fixed in the transverse borehole (2) of the medullary pin (1) so that they cannot rotate about the central axis (3).

12. The device of one of the claims 2 to 11, characterized in that the sheath (7) has an external thread (9).

13. The device of one of the claims 2 to 11, characterized in that the sheath (7) is configured as a blade (26).

14. The device of one of the claims 3 to 13, characterized and that the second sheath (25) has an external thread.

15. The device of one of the claims 3 to 13, characterized in that the second sheath (25) is constructed as a blade.

16. The device of one of the claims 1 to 15, characterized in that it includes expansion agents (8), which can be moved coaxially with the transverse borehole (2).

17. The device of claim 16, characterized and that the expansion agents (8) pass axially through the bone fixation agent (4) and, at the front end (6) of the bone fixation agents (4), have a cone (13), which tapers towards the rear end (5) of the bone fixation agents (4).

18. The device of claim 16, characterized in that the expansion agents (8) pass through the bone fixation agents (4) coaxially and the inner wall of the sheath (7) includes an inner cone (12), which expands towards the rear end (5) of the bone fixation agent (4), against which the expansion agents (8) can be shifted axially.

19. The device of one of the claims 2 to 18, characterized in that the shaft (18) can be connected with the sheath (7) by means of a press fit.

20. The device of one of the claim 16 to 19, characterized in that the expansion agents (8) can be shifted by means of a threaded connection between the shaft (18) and the expansion agents (8) coaxially with the central axis (3).

21. The device of one of the claims 16 to 19, characterized in that the expansion agents (8) can be shifted coaxially with the central axis (3) by means of a threaded connection between the sheath (7) and the expansion agents (8).

22. The device of one of the claims 1 to 21, characterized in that it includes additionally a second bone fixation agent (4).

23. An elastically expandable sheath (7), characterized in that it includes an external thread (9), a central borehole (11) coaxial with the central axis (3) and a slot (24), the slot (24) passing through the sheath (7) perpendicularly to the central axis (3) and penetrating into the sleeve (7) parallel to the central axis (3).
